(19) United States
(12) Patent Application Publication
(10) Pub. No.: US 2022/0273874 A1
(43) Pub. Date: Sep. 1, 2022

(54) METHOD AND SYSTEM OF FULLY-AUTOMATED ARTIFICIAL PANCREAS CONTROL FOR COUNTERACTING POSTPRANDIAL HYPERGLYCEMIA

(71) Applicant: University of Virginia Patent Foundation, Charlottesville, VA (US)

(72) Inventors: Marc D. BRETON, Charlottesville, VA (US); Patricio COLMEGNA, Charlottesville, VA (US)

(21) Appl. No.: 17/679,265

(22) Filed: Feb. 24, 2022

Related U.S. Application Data
(60) Provisional application No. 63/153,016, filed on Feb. 24, 2021.

(51) Int. Cl. A61M 5/172; G16H 20/17; G16H 40/63

(52) U.S. Cl. CPC A61M 5/1723; G16H 20/17; G16H 40/63; A61M 2230/201

(57) ABSTRACT

Provided are a method, system and computer-readable storage medium for fully-automated artificial pancreas (AP) control aimed at minimizing and/or preventing occurrence of hyperglycemia following an unannounced meal. Such control is modulated relative to a utilized insulin, the absorption level of which is basis for the control's aggressiveness in administering insulin. In this way, the control can, for increasing levels of absorption, be increasingly aggressive and thus avoid instances of hyperglycemia and hypoglycemia.

### CROSS-REFERENCE TO RELATED APPLICATION

This application claims the benefit of U.S. Provisional Application No. 63/153,016, filed Feb. 24, 2021, the entire contents of which is incorporated by reference herein.

### STATEMENT OF GOVERNMENT INTEREST

This invention was made with government support under Grant No. 1DP3DK106826-01 awarded by the National Institutes of Health, and under CTSA Grant No. UL1 RR024139 awarded by the National Center for Advancing Translational Science. The government has certain rights in the invention.

### FIELD OF THE DISCLOSURE

Disclosed embodiments relate to individual glucose control, and more specifically, to such control as enabled by use of fully-automated artificial pancreas (AP) control aimed at minimizing and/or preventing the occurrence of postprandial hyperglycemic events.

### BACKGROUND

In connection with discussion herein, superscript notations herein are to those references as delineated in the similarly entitled section herein. Additionally, the following listing of abbreviations shall apply, including: (T1D) Type 1 Diabetes, (AP) Artificial Pancreas, (SC) Subcutaneous, (IP) Intraperitoneal, (LIS) Insulin Lispro, (BC-LIS) BioChaperone Insulin Lispro, (DIA) Duration of Insulin Action, (PK) Pharmacokinetic, (PD) Pharmacodynamic, (GIR) Glucose Infusion Rate, (FDA) Food and Drug Administration, (UVA) University of Virginia, (MPC) Model Predictive Control, (LTI) Linear Time Invariant, (SOGMM) Subcutaneous Oral Glucose Minimal Model, (JOB) Insulin On Board, (USS) Unified Safety System, (CR) Insulin-to-Carbohydrate Ratio, (TDI) Total Daily Insulin, (LBGI) Low Blood Glucose Index, (HBGI) High Blood Glucose Index (gCHO) Grams of Carbohydrates.

Postprandial glycemia makes a substantial contribution to overall glycemic control in diabetes treatment. Unfortunately, meeting postprandial glycemic target values has been challenging due to slow absorption and action of subcutaneously injected insulins. Insulin secretion from a healthy β-cell is a highly dynamic process, where glucose is the main stimulator of insulin release, leading to the characteristic biphasic pattern consisting of a brief first phase of insulin secretion (˜10 minutes), followed by a sustained second phase. The earliest secreted insulin is a necessary element to offset the rapid rise in postprandial blood glucose. Unlike the rapid physiologic action of insulin after its release from a healthy β-cell, the maximum glucose lowering action from a subcutaneously injected insulin could be observed as late as 90 minutes to two hours after its injection.^{1,2} The underlying reasons for delay in insulin action are multifactorial, with chemical properties of insulin and factors concerning subcutaneous (SC) tissue being the principal contributors.³ Moreover, subcutaneously delivered insulin may pose additional glycemic risks due to its prolonged action (up to 6 h), potentially increasing the risk of late postprandial hypoglycemia. A single-hormonal artificial pancreas (AP) system optimizes insulin delivery in real time, every five minutes, based on changes in sensor glucose levels. While most current systems function best with a pre-meal insulin bolus (hybrid AP), a fully automated system would not benefit from this sharp and early increase in circulating insulin. Consequently, a fully automated AP insulin controller reacts to meals only after sensor glucose levels begin to rise. Besides, there is no insulin depot delivered in to the SC area as the insulin delivery is spread over hours in mini boluses. Therefore, the delay in insulin absorption and action is further exacerbated during fully automated AP, representing one of the main barriers to its implementation.^{4,5} Thus, the most common strategy is to define a single- or dual-hormone system with a hybrid controller, where feedforward insulin boluses are manually delivered at mealtimes, and the control law takes over the basal rate.⁶⁻¹¹ The drawback associated with this design is that manual priming requires user assessment of the total amount of carbohydrates for every meal, which is a burdensome and potentially inaccurate task for patients.^{12,13}

Other insulin delivery routes than SC delivery have been explored to generate more physiological plasma insulin profiles. For example, inhaled human insulin has shown tangible benefits with respect to SC insulin injections.¹⁴ However, this scheme also depends on prandial manual doses. Another alternative is to deliver insulin into the intraperitoneal (IP) space to minimize delays.¹⁵ For instance, fully automated AP delivery combined with IP insulin delivery has provided superior glucose control to that with SC insulin delivery in a short demonstration study.¹⁶ Nevertheless, this approach's clinical application is still limited by its inherent costs and risk profile.¹⁷

Although fully-automated AP control has been successfully deployed in clinical studies,¹⁸⁻²⁴ there is an undeniable compromise between the controller's aggressiveness and insulin stacking due to the extended duration of insulin action (DIA). An ideal insulin analogue should mimic the pharmacokinetic (PK) and pharmacodynamic (PD) profiles of endogenous insulin to optimize exogenous insulin treatment. Rapid acting insulin analogs with faster PKPD profiles have been introduced recently towards this goal,²⁵⁻²⁷ but a significant unmet need for more rapid insulin absorption that provides superior postprandial glucose control remains, particularly as new AP technology enters clinical care.^{1,28}

In these regards, it would be advantageous to provide a manner of avoiding glucose dysregulation via a fully-automated AP control that can regulate glucose levels similarly as in the case of optimal hybrid AP implementations.

### SUMMARY

It is to be understood that both the following summary and the detailed description are exemplary and explanatory and are intended to provide further explanation of the present embodiments as claimed. Neither the summary nor the description that follows is intended to define or limit the scope of the present embodiments to the particular features mentioned in the summary or in the description. Rather, the scope of the present embodiments is defined by the appended claims.

Embodiments may include a method, system, computer-readable storage medium regarding artificial pancreas (AP) control for attaining normoglycemia following an unannounced meal of a subject, including (a) for a selected insulin, determining at least a corresponding absorption level, (b) based on a corresponding duration of insulin action (DIA) in dependence on the at least a corresponding absorption level, adjusting the control to change, from a datum, at least a first control parameter penalizing insulin deviation from basal rate and at least a second control parameter representing a difference between two consecutive insulin infusions, and (c) in response to a detected increase in glucose, infusing the selected insulin in accordance with the adjusted control.

In response to the selected insulin comprising a corresponding absorption level that is higher as against a corresponding absorption level for a non-selected insulin, the at least a first control parameter decreases and the at least a second control parameter increases.

Embodiments can increase the infusing based on the at least a first control parameter and the at least a second control parameter.

The infusing the selected insulin can be performed to align insulin and meal rates of appearance.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. Embodiments herein will be more particularly described in conjunction with the following drawings wherein:

FIG. 1 illustrates mean PK profiles for different values of a, with the circumferential indicator at 1 h representing administration of a 0.2 U/kg insulin bolus and crosses (x) representing peak levels;

FIG. 2 illustrates mean times to peak insulin levels for different values of a, with vertical lines representing standard error bars;

FIG. 3 illustrates mean GIR profiles for different values of a, with the circumferential indicator at 1 h representing administration of a 0.2 U/kg insulin bolus and crosses (x) representing peak levels;

FIG. 4 illustrates mean times to peak GIR levels for different values of a, with vertical lines representing standard error bars;

FIG. 5 illustrates mean DIA (fitted exponential function) for different values of a;

FIG. 6 illustrates a closed-loop response obtained relative to administration of LIS for an announced meal;

FIG. 7 illustrates a closed-loop response obtained relative to administration of LIS and different levels of fully-automated AP aggressiveness for an unannounced meal (with boundaries of the filled areas representing the 5^{th} and 95^{th} percentiles);

FIG. 8 illustrates a closed-loop response obtained relative to administration of α-insulin and different levels of fully-automated AP aggressiveness for an unannounced meal (with boundaries of the filled areas representing the 5^{th} and 95^{th} percentiles);

FIG. 9 illustrates comparison between mean percentages of time <70 mg/dL and >180 mg/dL relative to use of LIS and α-insulin for different levels of fully-automated AP aggressiveness (with boundaries of the filled areas representing the 5^{th} and 95^{th} percentiles);

FIG. 10 illustrates a comparison of glucose trajectories obtained with LIS and a baseline hybrid AP control relative to fully-automated AP control using α-insulin and aggressiveness for α=1;

FIG. 11 illustrates a comparison of glucose trajectories obtained with LIS and a baseline hybrid AP control relative to fully-automated AP control using α-insulin and aggressiveness for α=2;

FIG. 12 illustrates a comparison of glucose trajectories obtained with LIS and a baseline hybrid AP control relative to fully-automated AP control using α-insulin and aggressiveness for α=3; and

FIG. 13 illustrates mean glucose rate of appearance (Rₐ) versus mean insulin rate of appearance (Rᵢ) relative to basal value from each of FIG. 7 (at “p”) and FIG. 8 (at “q”).

### DETAILED DESCRIPTION

The present disclosure will now be described in terms of various exemplary embodiments. This specification discloses one or more embodiments that incorporate features of the present embodiments. The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. The skilled artisan will appreciate that a particular feature, structure, or characteristic described in connection with one embodiment is not necessarily limited to that embodiment but typically has relevance and applicability to one or more other embodiments.

In the several figures, like reference numerals may be used for like elements having like functions even in different drawings. The embodiments described, and their detailed construction and elements, are merely provided to assist in a comprehensive understanding of the present embodiments. Thus, it is apparent that the present embodiments may be carried out in a variety of ways, and does not require any of the specific features described herein. Also, well-known functions or constructions are not described in detail since they would obscure the present embodiments with unnecessary detail.

The description is not to be taken in a limiting sense, but is made merely for the purpose of illustrating the general principles of the present embodiments, since the scope of the present embodiments are best defined by the appended claims.

It should also be noted that in some alternative implementations, the blocks in a flowchart, the communications in a sequence-diagram, the states in a state-diagram, etc., may occur out of the orders illustrated in the figures. That is, the illustrated orders of the blocks/communications/states are not intended to be limiting. Rather, the illustrated blocks/communications/states may be reordered into any suitable order, and some of the blocks/communications/states could occur simultaneously.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedure, Section 2111.03.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. The word “exemplary” is used herein to mean “serving as an example, instance, or illustration.” Any embodiment described herein as “exemplary” is not necessarily to be construed as preferred or advantageous over other embodiments. Additionally, all embodiments described herein should be considered exemplary unless otherwise stated.

It should be appreciated that any of the components or modules referred to with regards to any of the embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Ranges may be expressed herein as from “about” or “approximately” one particular value and/or to “about” or “approximately” another particular value. When such a range is expressed, other exemplary embodiments include from the one particular value and/or to the other particular value.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

Herein, we inspect a degree to which the analogue insulin lispro (LIS) glucodynamic action can be accelerated to safely increase a fully-automated AP controller's aggressiveness in a SC AP with a model predictive control (MPC) law. To this end, we leverage the UVA/Padova simulator²⁹ to test the performance of the proposed controller in scenarios that include both announced and unannounced meals and different synthetic insulins.

As such, discussed below are (a) a model of insulin pharmacokinetics, (b) in silico generation of faster insulin analogues, and (c) model predictive control (MPC) for regulating blood glucose level.

Model of Insulin Pharmacokinetics

We consider the two-compartment PK model of SC fast-acting insulin that was presented in³⁰ and later updated in:³¹

İ_{sc1}(t)=−(k_{α1}+k_{d})I_{sc1}(t)+u(t−τ) (1)

İ_{sc2}(t)=−k_{α2}I_{sc2}(t)+k_{d}I_{sc1}(t) (2)

Rᵢ(t)=k_{α1}I_{sc1}(t)+k_{α2}I_{sc2}(t) (3),

where I_{sc1} and I_{sc2} [pmol/min] are, respectively, the amounts of monomeric and non-monomeric insulin in the subcutaneous space, k_{α1} and k_{α2} [l/min] are the corresponding rate constants of absorption into plasma, k_{d} [l/min] is the diffusion rate from non-monomeric to monomeric state, u [pmol/kg/min] is the exogenous insulin infusion rate, r [min] is a subject-specific input delay, and Rᵢ [pmol/kg/min] is the rate of insulin absorption into plasma. In Ref.³¹, the PK model is identified using insulin data collected from 116 adult subjects with type 1 diabetes (T1D) who underwent a SC injection of LIS. Individual sets of PK parameters were extracted from parameter distributions obtained from model identification that were then randomly assigned to each in silico subject of the simulator. Analysis of population sets indicate that all PK parameters follow a lognormal probability distribution and are uncorrelated from each other and from the other parameters of the UVA/Padova model.

In Silico Generation of Faster Insulin Analogues

The model described by equations (1)-(3) is a second-order time-delay linear time-invariant (LTI) system with the following transfer function:

$${G}_{{i}_{sc}} ( s ) = \frac{{k}_{a 1} s + {k}_{a 2} ( {k}_{a 1} + {k}_{d} )}{( s + {k}_{d} + {k}_{a 1} ) ( s + {k}_{a 2} )} {e}^{- τ s} \qquad ( 4 )$$

As shown, Gᵢ_{_{sc}} has two poles located at p₁=−(k_{d}+k_{α1}) and P₂=−k_{α2}. According to the parameter estimates reported in³¹, the mean value of k_{α1} is close to zero and negligible with respect to the mean values of both k_{d} and k_{α2}. Thus, Gᵢ_{_{sc}}(s) can be approximated as:

$${G}_{{i}_{sc}} ( s ) ≅ {\tilde{G}}_{{i}_{sc}} ( s ) = \frac{{k}_{a 2} {k}_{d}}{( s + {k}_{d} ) ( s + {k}_{a 2} )} {e}^{- τ s} \qquad ( 5 )$$

In order to define faster insulin analogues, we accelerate the insulin absorption from the subcutaneous tissue by manipulating only the poles of \tilde{G}ᵢ_{_{sc}}(s) while keeping the other parameters unchanged. To this end, lognormal distributions were fitted to the vectors of parameters k_{d} and k_{α2} associated with LIS, and new sets were sampled from the fitted distributions, but with their mean values modified by a factor of α>1, where α represents insulin absorption level (i.e., extent and/or rate).

The bandwidth of a system is commonly defined as the lowest frequency satisfying −3 dB from its gain at zero frequency. Accordingly, if the average bandwidth of the PK model for LIS is ωₗ, then the average bandwidth for the α-insulin analogue will be ω_{f}=αωₗ. In this way, as a increases in magnitude, the faster the insulin analogue becomes.

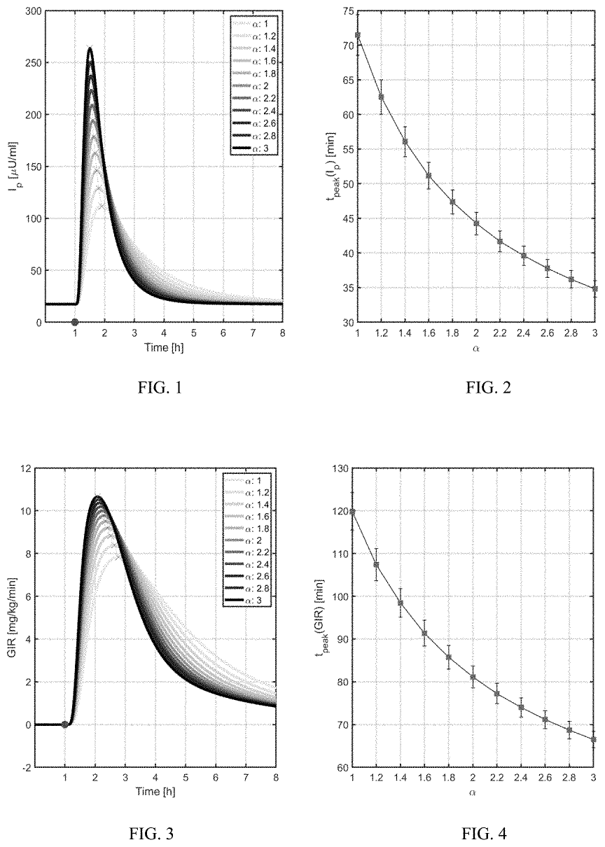

In order to determine the PKPD properties of the α-insulins, a euglycemic clamp was performed in simulation. In this in silico procedure, a 0.2 U/kg single dose of α-insulin was administered to each of the 100 in silico adults of the UVA/Padova simulator and the simulated intravenous glucose infusion rates (GIR) were automatically adjusted by means of a proportional controller that maintained the glucose levels close to the basal values. FIGS. 1 and 2 illustrate the PK and GIR profiles, respectively, for different values of a.

In Ref.³², this euglycemic glucose clamp is carried out on 38 adult patients with T1D to compare the PKPD properties of LIS and ultra-rapid BioChaperone LIS³³ (BC-LIS). Results demonstrate that times to maximum insulin levels and GIR occur 20 and 30 minutes earlier, respectively, with BC-LIS. Bearing this in mind, and for merely illustrative purposes, we can associate BC-LIS with α≅1.6 in our approach. That is, it is contemplated herein that one or more types of insulin can be relatively compared to arrive at a measure for α.

Model Predictive Control for Regulating Blood Glucose Level

To assess the impact of faster insulins on the performance of an AP, we consider an originally hybrid MPC law as a baseline. This control strategy has been published by the authors elsewhere,³⁴ and a summary of its formulation is provided below.

The proposed MPC is based on the so-called Subcutaneous Oral Glucose Minimal Model (SOGMM).³⁶ To embed this model into the MPC formulation, it is first linearized at the steady state given by the subject-specific insulin basal rate u_{b} [mU/min] and a blood glucose setpoint of 120 mg/dl, and later discretized with a sampling period Tₛ=5 min. In this way, a triplet (A, B, C) that describes the insulin-glucose dynamics is obtained.

Let u,y∈ [symbol] denote the insulin and glucose deviations from steady state, and x∈ [symbol] ⁿ, the model state vector. Denoting the prediction and control horizons by Nₚ and N_{c}, respectively, we formulate the following MPC problem that is solved at each step k:

$$[ {\tilde{u}}_{k} , {\tilde{η}}_{k} ] = {arg min}_{{\tilde{u}}_{k} , {\tilde{η}}_{k}} J ( {x}_{k} , {\tilde{u}}_{k} , {\tilde{η}}_{k} ) \qquad ( 10 )$$

$$with cost function$$

$$J ( \cdot ) = {\sum }_{j - k + 1}^{k + {V}_{o}} [ {Q ( {y}_{j} - {r}_{j} )}^{2} + {κη}_{j - 1}^{2} ] + {\sum }_{j = k}^{k + N - 1} λΔ {u}_{j}^{2} \qquad ( 11 )$$

$$subject to$$

$${x}_{k} = {\hat{x}}_{k} \qquad ( 12 )$$

$$\begin{matrix}{x}_{j - 1} = {Ax}_{j} + {Bu}_{j} & ∀ j ∈ {ℕ}_{k}^{k + {N}_{o} - 1}\end{matrix} \qquad ( 13 )$$

$$\begin{matrix}{y}_{j} = {Cx}_{J} & ∀ j ∈ {ℕ}_{k}^{k + {N}_{y}}\end{matrix} \qquad ( 14 )$$

$$\begin{matrix}{u}_{min} \leq {u}_{i} \leq {u}_{max} & ∀ j ∈ {ℕ}_{k}^{k + {N}_{i} - 1}\end{matrix} \qquad ( 15 )$$

$$\begin{matrix}Δ {u}_{j} \leq Δ {u}_{max} & ∀ j ∈ {ℕ}_{k}^{k + {N}_{v} - 1}\end{matrix} \qquad ( 16 )$$

$$\begin{matrix}{y}_{min} - {y}_{j} \leq {η}_{j - 1} & ∀ j ∈ {ℕ}_{k + 1}^{k + {N}_{o}}\end{matrix} \qquad ( 17 )$$

$$\begin{matrix}{η}_{j} \geq 0 & ∀ j ∈ {ℕ}_{k}^{k + {N}_{p} - 1}\end{matrix} \qquad ( 18 )$$

$${r}_{j} = \{ \begin{matrix}{y}_{k} \cdot {e}^{- ( j - k ) / x} , & {y}_{k} \geq 0 \\ 0 , & otherwise\end{matrix} ∀ j ∈ {ℕ}_{k}^{k + {N}_{y} - 1} \qquad ( 19 )$$

Predictions of the insulin-glucose dynamics are made using the obtained state-space realization (A, B, C) (Eqns. 13,14) with the initial state xₖ estimated by means a Kalman filter (Eqn. 12). Eqns. (15) and (16) enforce that the insulin infusion lies in the interval [uₘᵢₙ, uₘₐₓ], and the difference between two consecutive insulin infusions is not higher than Δuₘₐₓ, respectively. Eqns. (17) and (18) enforce a soft constraint on the glucose lower bound yₘᵢₙ (hypoglycemic threshold). Three positive scalars are included in the cost function: (i) κ that penalizes control actions that lead to low glucose levels, (ii) A that weights Au, and (iii) Q that penalizes glucose deviations from the asymmetric, time-varying, exponential reference signal r.³⁷

Sequence ũₖ*={uₖ*, . . . , u_{k+N}_{_{c}}₋₁*} contains the optimal control policy and sequence \tilde{η}ₖ*={ηₖ*, . . . , η_{k+N}_{ₚ}₋₁*} the optimal slack variables associated with the soft constraint. In this formulation, the control signal at step k is defined as the first element of ũₖ*, i.e., uₖ=uₖ*. In order to minimize the risk of hypoglycemia the controller is combined with an auxiliary module, the so-called Unified Safety System (USS Virginia) that enforces a limit to basal injections when low glucose values are predicted.³⁸

Relative to this baseline MPC, the approach herein contemplates two detuning stages as outlined below, and including (a) detuning of MPC controller aggressiveness (Q), and (b) detuning of λ and Δuₘₐₓ.

Detuning of MPC Controller Aggressiveness

In a hybrid AP approach, it is assumed that meal disturbances are mostly mitigated by feedforward insulin boluses that are delivered at mealtimes. In this case, the user needs to calculate the prandial dose based on, among other factors, the meal size in grams of carbohydrates (gCHO) and his/her insulin-to-carbohydrate ratio (CR) in gCHO/U. In order to avoid a controller overreaction to postprandial glucose excursions, the scalar weight Q that penalizes glucose deviations from target (see the Appendix for a description of Q in the MPC formulation) is detuned, according to the present embodiments, as follows:

$$Q ( IOB ) = \{ \begin{matrix}{Q}_{0} & if IOB < 0 \\ \frac{{β}_{1} \cdot ( 1 - {β}_{2} ) \cdot {Q}_{0}}{{β}_{2} \cdot TDI} \cdot IOB + {Q}_{0} & if IOB ∈ [ 0 , TDI / {β}_{1} ] \\ {Q}_{0} / {β}_{2} & if IOB > TDI / {β}_{1}\end{matrix} \qquad ( 6 )$$

where Q₀ is the value of Q at steady state, TDI [U/day] denotes the subject-specific total daily insulin requirement, IOB [U] is the insulin-on-board relative to the expected IOB from basal delivery, and β₁ and β₂ are tuning parameters. In this way, when a meal bolus is delivered, the IOB estimate will have a peak, resulting in desensitizing the controller to glucose deviations from reference. In this regard, the higher β₁ and β₂ are in magnitude, the less responsive the controller can be at mealtimes.

Detuning of λ and Δuₘₐₓ

Long delays in insulin peak and duration substantially limit the achievable sensitivity of an AP to glucose deviations. This is the case since an aggressive control law can lead to late hypoglycemia due to insulin stacking. Here, we propose to re-tune the controller's aggressiveness based on the dynamics of the insulin analogue: the faster the insulin analogue is, the more aggressive the controller can be. To this end, the average DIA was calculated for several α-insulins, and fitted using a nonlinear least-squares approach by the following exponential function derived from the structure of Eqn. (5):

DIA(α)=γ₁e^{γ}^{₂}^{α}+γ₃e^{γ}^{₄}^{α} (7)

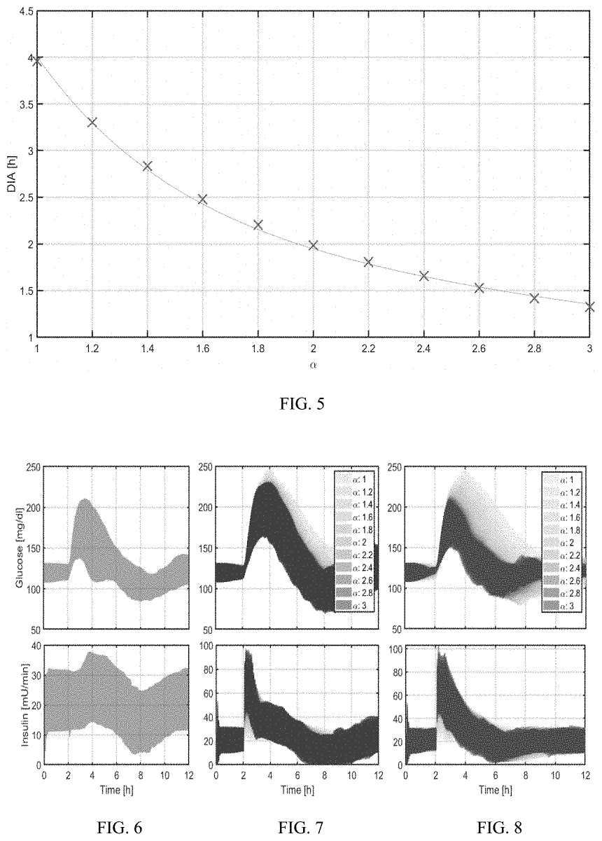

with γ₁=13.83, γ₂=−2.05, γ₃=2.89, and γ₄=−0.26 (see FIG. 5). To make the controller more aggressive for faster insulins, design parameters λ and Δuₘₐₓ are now defined as functions of the DIA as follows:

$$λ ( DIA ) = \{ \begin{matrix}{ψ}_{1} {e}^{{ψ}_{2} \cdot DIA} / {u}_{b} & if DIA < 4 h \\ {ψ}_{1} {e}^{{ψ}_{2} \cdot 4} / {u}_{b} & otherwise\end{matrix} \qquad ( 8 )$$

$${Δu}_{max} ( DIA ) = \{ \begin{matrix}- {ψ}_{3} \cdot DIA + {ψ}_{4} & if DIA < 4 h \\ - {ψ}_{3} \cdot 4 + {ψ}_{4} & otherwise\end{matrix} \qquad ( 9 )$$

Numerical values of the tuning parameters ψᵢ with i={1, . . . , 4} along with all the other parameters for the MPC are reported in Table 1 below. In this way, when the controller commands LIS (DIA=4 h), first control parameter λ, which penalizes insulin deviations from basal rate, and second control parameter Δuₘₐₓ, which represents the difference between two consecutive insulin infusions (each of the parameters being set forth above), are set to their default values (λ₀, Δuₘₐₓ_{₀}) (i.e., datum). However, first control parameter λ decreases and second control parameter Δuₘₐₓ increases as insulin is accelerated, i.e., as α is increased, thereby allowing the controller to take more aggressive actions. That is, the controller of a fully-automated AP can, when compared to administration for a non-accelerated insulin (i.e., λ of lesser value), deliver SC administration more aggressively for an increasing α and decreasing DIA. Statistical comparisons between results obtained with the hybrid controller and LIS, and the fully-automated controller and α-insulin were determined using a t-test of significance for means and a Mann-Whitney U-test for medians.

TABLE 1
Tuning parameters of the MPC law.

| Parameter | Value | Parameter | Value |
|---|---|---|---|
| Nₚ | 24 | γₘᵢₙ | 70 mg/dl |
| N_{c} | 18 | τᵣ | 5 |
| Q₀ | 10 | β₁ | 20 |
| κ | 100 | β₂ | 1000 |
| λ₀ | ψ₁e^{ψ}^{₂}^{·4}/u_{b} | ψ₁ | 18 |
| uₘᵢₙ | −u_{b} | ψ₂ | 1.125 |
| uₘₐₓ | 1000 mU/ml-u_{b} | ψ₃ | 25 |
| Δuₘₐₓ_{₀} | 50 mU/ml | ψ₄ | 150 |

Results

Herein, a framework for testing of the method is presented to evidence the impact of accelerating insulin absorption and action on post-meal hyperglycemia mitigation using a fully-automated AP controller. To this end, 12 hour simulations that include different α-insulins and one (un)announced meal challenge are performed considering the proposed MPC as the control law. In order to test robustness with respect to inter-subject variability, simulations are run for all 100 adult subjects of the UVA/Padova simulator. Outcomes are computed over the 8 hours following the meal so as to capture both early hyperglycemia and late hypoglycemia. Time responses are depicted in FIGS. 6-8 and numerical results, including average glucose values, time in ranges, and risk indices, are tabulated in Table 2 below. Both Low Blood Glucose Index (LBGI) and High Blood Glucose Index (HBGI)³⁵ have been included in this analysis to quantify the risks of hypo- and hyperglycemia obtained with each closed-loop strategy.

Glycemic Control Using a Hybrid Approach

To define a baseline of hybrid glucose control, a first set of simulations is carried out with LIS and meal announcement, i.e., delivering feedforward meal-boluses at mealtimes. Given the meal-size M=50 gCHO and the subject's CR, the bolus size is calculated as M/CR. Average time responses are depicted in FIG. 6 and numerical results are tabulated in the first set of columns of Table 2 below.

Glycemic Control with Unannounced Meals

In this control, the prandial bolus is eliminated and the controller's aggressiveness is gradually increased. To this end, we tune the MPC using Eqns. (8)-(9), but keep using LIS in the simulations. That is, α is only used to define the controller's aggressiveness, but not to accelerate the insulin analogue. Average time responses are illustrated in FIG. 7 and numerical results are tabulated in the second set of columns of Table 2 Error! Reference source not found. below. Note that not only the mean percentage of time in the range [70, 180] mg/dl increases (70.1, 95% CI [66.9, 73.4] for α=1 vs 81.4, [78.6, 84.3] for α=3, P<0.05), but also the mean percentage of time below 70 mg/dl (0.0, [0.0, 0.0] for α=1 vs 1.4, [0.7, 2.8] for α=3, P<0.05). The same situation is observed with respect to the risk indices: LBGI 0.05, [0.03, 0.10] for α=1 vs 0.52, [0.36, 0.75] for α=3, P<0.05; HBGI 5.29, [4.88, 5.71] for α=1 vs 3.28, [3.00, 3.56] for α=3, P<0.05.

The final step is to repeat these simulations but switching from LIS to the corresponding accelerated α-insulin. Results are illustrated in FIG. 8, and the third set of columns of Table 2 below. In this case, a more marked increase in time in range is detected (70.1, [66.9, 73.4] for α=1 vs 94.1 [92.6, 95.6] for α=3, P<0.05), with a slight non-significant increase in time below 70 mg/dl (0.0, [0.0, 0.0] for α=1 vs 0.4, [0.1, 1.4] for α=3, P=0.13). Similarly for the risk indices: LBGI 0.05, [0.03, 0.10] for α=1 vs 0.14, [0.07, 0.30] for α=3, P=0.09; HBGI 5.29, [4.88, 5.71] for α=1 vs 1.66, [1.52, 1.80] for α=3, P<0.05.

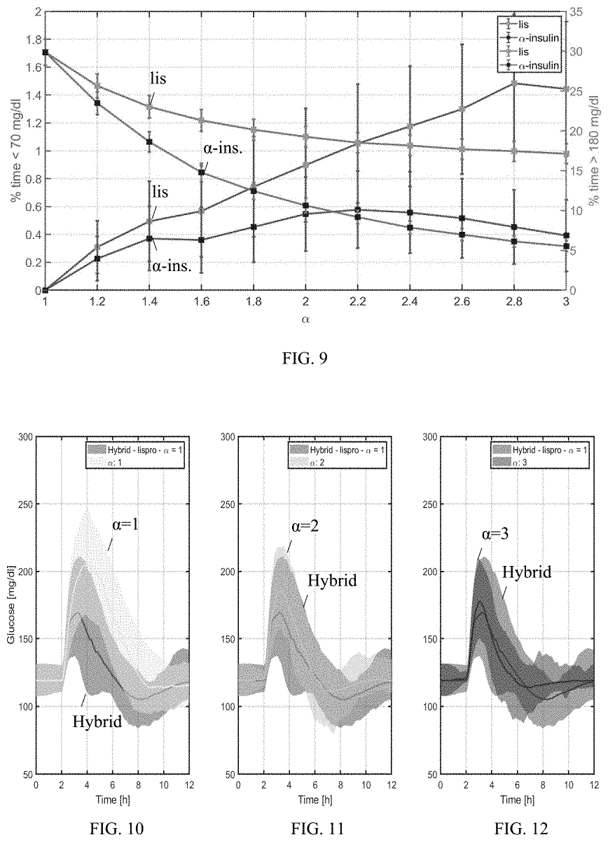

FIG. 9 indicates how the percentages of time <70 mg/dl and >180 mg/dl evolve with LIS and the α-insulin analogues as the controller's aggressiveness is increased. Glucose trajectories from FIGS. 6-8 are overlapped in FIGS. 10-12 to facilitate the comparison between both the hybrid and reactive, i.e., fully-automated, AP approaches. Results indicate that non-significant difference between medians is obtained for α≥2.4. In this way, for a reactive AP that does not rely on manual insulin boluses at mealtimes to match the glucose control performance achievable by its hybrid version, times to maximum insulin levels and GIR obtained with BC-LIS (α≅1.6) have to occur 10 and 15 minutes earlier, respectively, according to FIGS. 1 and 2.

As is demonstrated, if the acceleration of the insulin analogue is not accompanied by an increase in the controller's aggressiveness, then the benefits of faster insulins in glucose control are less noticeable. For instance, if a is only used to accelerate the insulin analogue, but not to increase the controller's aggressiveness, a less marked increase in time in range is observed (70.1, [66.9, 73.4] for α=1 vs 79.5 [76.5, 82.4] for α=3, P<0.05), although with no increase in time below 70 mg/dl (0.0, [0.0, 0.0] for α=1 vs 0.0, [0.0, 0.0] for α=3).

### DISCUSSION

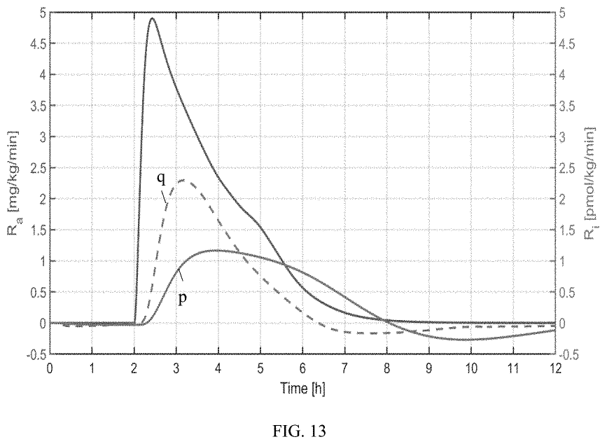

Hybrid AP systems rely on feedforward insulin boluses to manage postprandial glucose excursions and on the glucose controller to maintain normoglycemia by modulating the basal insulin delivery. Users play a key role in this scheme since carbohydrate counting is cornerstone for meal insulin bolus calculation. Although this method reduces the stress on the controller, it is burdensome for patients and prone to human errors that may affect the achievable glucose control performance. One alternative is to eliminate the meal announcement from the control structure and tune the controller to be more reactive to glucose deviations. The longer the time to peak, the more sensitive the controller needs to be to alleviate postprandial hyperglycemia. As shown in FIGS. 7-8, if the baseline hybrid MPC is used without meal boluses (α=1), large glucose excursions are manifested since the controller is purposely designed to perform only slight modifications to the basal rate. As such, increasing the controller's aggressiveness to deliver an insulin ‘kick’ at mealtimes is not appropriate relative to a given insulin analogue, since doing so may contribute to the risk of hypoglycemic values towards the end of the meal-response. By contrast, FIGS. 6-8 reveal that in response to the controller's aggressiveness being increased in combination with faster acting insulins, both a faster descend from peak to trough and a superior protection to late hypoglycemia are easily discernible. Above all, the controller should align the insulin and meal rates of appearance for effective postprandial glucose control (see FIG. 13). With this in mind, faster insulin analogues can be a critical means to achieve that goal in a SC fully-automated AP approach. In any case, the proposed control strategy can still be applied in a hybrid scheme, since the controller is de-tuned for high IOB values (Eqn. (6)).

TABLE 2
Comparison Between Numerical Results Related to Each Set of Closed-Loop Simulations

| | Announced meal | | Unannounced meal | | | | | | Unannounced meal | | | | | |
|---|---|---|---|---|---|---|---|---|---|---|---|---|---|---|
| | Insolin | | | | | | | | | | | | | |
| | [symbol] | | [symbol] | | | | | | α-insolin | | | | | |
| | α | | | | | | | | | | | | | |
| | 1 | | 1 | | 2 | | 3 | | 1 | | 2 | | 3 | |
| | Mean | Median | | Median | | Median | | Median | | Median | | Median | | Median |
| | | [JQR] | Mean | [JQR] | Mean | [JQR] | Mean | [JQR] | Mean | [JQR] | Mean | [JQR] | Mean | [JQR] |
| Average | 130 | 128 | 157 | 155 | [symbol] | 140 | 138 | 138 | 137 | 135 | 135 | 135 | [symbol] | 130 |
| glucose [mg/dl] | | [ [symbol] ] | | [18] | | [12] | | [11] | | [18] | | [ [symbol] ] | | [6] |
| % time < 50 | 0 | 0 | 0 | 0 | 0.1 | 0 | 0.2 | 0 | 0 | 0 | 0.1 | 0 | 0.3 | 0 |
| mg/dl | | [0] | | [0] | | [0] | | [0] | | [0] | | [0] | | [0] |
| % time < 70 | 0.1 | 0 | 0 | 0 | 0.9 | 0 | [symbol] | 0 | 0 | 0 | 0. [symbol] | 0 | 0.4 | 0 |
| mg/dl | | [0] | | [0] | | [0] | | [0] | | [0] | | [0] | | [0] |
| % time in | 92.9 | [symbol] | 70.1 | 67.0 | 73.9 | 77.8 | 81.4 | 70.4 | 70.1 | 87.0 | 88.8 | 88.7 | 94.1 | 100 |
| [70.180] mg/dl | | [ [symbol] ] | | [18] | | [21] | | [24] | | [18] | | [18] | | [11] |
| % time > 180 | 7.0 | 0 | 29.9 | 33.0 | 19.2 | 22.2 | 17.2 | 20.3 | 23.9 | 33.0 | 10.0 | 11.3 | 5.5 | 0 |
| mg/dl | | [ [symbol] ] | | [18] | | [21] | | [23] | | [18] | | [18] | | [11] |
| % time > 250 | 0 | 0 | 9.6 | 0 | 0.3 | 0 | 0.2 | 0 | 0.6 | 0 | 0 | 0 | 0 | 0 |
| mg/dl | | [0] | | [0] | | [0] | | [0] | | [0] | | [0] | | [0] |
| LGBI | 0.19 | 0.07 | 9.05 | 0 | [symbol] | 0.11 | 0.53 | 0.18 | 0.03 | 0 | 0.21 | 0.01 | 0.14 | 0 |
| | | [0] | | [0] | | [0] | | [1] | | [0] | | [0] | | [0] |
| HBGI | 1.83 | 1.54 | 3.29 | 3.26 | 3.56 | 3.57 | 3.28 | 3.28 | 3.29 | [symbol] | 2.4 | 2.40 | 1.66 | 1.63 |
| | | [2] | | [3] | | [2] | | [2] | | [3] | | [1] | | [1] |

[symbol]

indicates data missing or illegible when filed

Thus, as can be understood from the above, embodiments herein can implement a fully-automated AP control for real subjects (i.e., patients) to minimize and/or prevent instances of hyperglycemia and hypoglycemia following an unannounced meal. In these regards, such control can utilize one or more insulin types whereby at least an absorption level thereof can influence the aggressiveness with which is insulin is administered to a subject. In this way, the AP control of embodiments herein can achieve glycemic performance similar to that of optimal hybrid AP control in use of prandial insulin boluses.

### REFERENCES

The devices, systems, apparatuses, modules, compositions, computer program products, non-transitory computer readable medium, models, algorithms, and methods of various embodiments disclosed herein may utilize aspects (devices, systems, apparatuses, modules, compositions, computer program products, non-transitory computer readable medium, models, algorithms, and methods) disclosed in the following references, applications, publications and patents and which are hereby incorporated by reference herein in their entirety, and which are not admitted to be prior art with respect to the present embodiments by inclusion in this section:
1. Cengiz E. Undeniable need for ultrafast-acting insulin: The pediatric perspective. J Diabetes Sci Technol. 2012; 6(4):797-801.
2. Cengiz E, Bode B, Van Name M, Tamborlane W. Moving toward the ideal insulin for insulin pumps. Expert Rev Med Devices. 2016; 13(1):57-69.
3. Gradel A K J, Porsgaard T, Lykkesfeldt J, et al. Factors Affecting the Absorption of Subcutaneously Administered Insulin: Effect on Variability. J Diabetes Res. 2018; 2018. doi:10.1155/2018/1205121
4. Steil G, Panteleon A, Rebrin K. Closed-loop insulin delivery—the path to physiological glucose control. Adv Drug Deliv Rev. 2004; 56(2):125-144.
5. Bequette B. Challenges and recent progress in the development of a closed-loop artificial pancreas. Annu Rev Control. 2012; 36(2):255-266.
6. Ly T, Roy A, Grosman B, Shin J, Campbell A, Monirabbasi S. Day and night closed-loop control using the integrated Medtronic hybrid closed-loop system in type 1 diabetes at diabetes camp. Diabetes Care. 2015; 38(7):1205-1211.
7. Bally L, Thabit H, Kojzar H, Mader J, Qerimi-Hyseni J, Hartnell S. Day-and-night glycaemic control with closed-loop insulin delivery versus conventional insulin pump therapy in free-living adults with well controlled type 1 diabetes: An open-label, randomised, crossover study. Lancet Diabetes Endocrinol. 2017; 5(4):261-270.
8. Forlenza G, Deshpande S, Ly T, Howsmon D, Cameron F, Baysal N. Application of zone model predictive control artificial pancreas during extended use of infusion set and sensor: A randomized crossover-controlled home-use trial. Diabetes Care. 2017; 40(8):1096-1102.
9. Kovatchev B, Cheng P, Anderson S M, et al. Feasibility of long-term closed-loop control: A multicenter 6-month trial of 24/7 automated insulin delivery. Diabetes Technol Ther. 2017; 19(1):18-24.
10. El-Khatib F, Balliro C, Hillard M, Magyar K, Ekhlaspour L, Sinha M. Home use of a bihormonal bionic pancreas versus insulin pump therapy in adults with type 1 diabetes: A multicentre randomised crossover trial. Lancet. 2017; 389(10067):369-380.
11. Haidar A, Messier V, Legault L, Ladouceur M, Rabasa-Lhoret R. Outpatient 60-hour day-and-night glucose control with dual-hormone artificial pancreas, single hormone artificial pancreas, or sensor-augmented pump therapy in adults with type 1 diabetes: An open-label, randomised, crossover, controlled trial. Diabetes Obes Metab. 2017; 19(5):713-720.
12. Bishop F, Maahs D, Spiegel G, Owen D, Klingensmith G. The carbohydrate counting in adolescents with type 1 diabetes (CCAT) study. Diabetes Spectr. 2009; 22(1):56-62.
13. Brazeau A, Mircescu H, Desjardins K, Leroux C, Strychar I, Ekoé J. Carbohydrate counting accuracy and blood glucose variability in adults with type 1 diabetes. Diabetes Res Clin Pract. 2013; 99(1):19-23.
14. Visentin R, Giegerich C, Jager R, Dahmen R, Boss A. Improving efficacy of inhaled technosphere insulin (Afrezza) by postmeal dosing: In-silico clinical trial with the University of Virginia/Padova type 1 diabetes simulator. Diabetes Technol Ther. 2016; 18(9):574-585.
15. Renard E. Insulin Delivery Route for the Artificial Pancreas: Subcutaneous, Intraperitoneal, or Intravenous? Pros and Cons. J Diabetes Sci Technol. 2008; 2(4):735-738.
16. Dassau E, Renard E, Place J, Farret A, Pelletier M. Intraperitoneal insulin delivery provides superior glycaemic regulation to subcutaneous insulin delivery in model predictive control-based fully-automated artificial pancreas in patients with type 1 diabetes: A pilot study. Diabetes Obes Metab. 2017; 19(12):1698-1705.
17. Jeandidier N, Boullu S, Busch-Brafin M, et al. Comparison of antigenicity of Hoechst 21PH insulin using either implantable intraperitoneal pump or subcutaneous external pump infusion in type 1 diabetic patients. Diabetes Care. 2002; 25(1):84-88.
18. Steil G, Rebrin K, Darwin C, Hariri F, Saad M. Feasibility of automating insulin delivery for the treatment of type 1 diabetes. Diabetes. 2006; 55(12):3344-3350.
19. Dassau E, Zisser H, Harvey R, Percival M, Grosman B, Bevier W. Clinical evaluation of a personalized artificial pancreas. Diabetes Care. 2013; 36(4):801-809.
20. Blauw H, van Bon A, Koops R, DeVries J, Consortium on behalf of the P. Performance and safety of an integrated bihormonal artificial pancreas for fully automated glucose control at home. Diabetes Obes Metab. 2016; 18(7):671-677.
21. Cameron F, Ly T, Buckingham B, et al. Closed-loop control without meal announcement in type 1 diabetes. Diabetes Technol Ther. 2017; 19(9):527-532.
22. Reddy M, Herrero P, Sharkawy M, Pesl P, Jugnee N, Pavitt D. Metabolic control with the bio-inspired artificial pancreas in adults with type 1 diabetes: A 24-hour randomized controlled crossover study. J Diabetes Sci Technol. 2015; 10(2):405-413.
23. Turksoy K, Hajizadeh I, Samadi S, Feng J, Sevil M, Park M. Real-time insulin bolusing for unannounced meals with artificial pancreas. Control Eng Pract. 2017; 59:159-164.
24. Sanchez-Peña R, Colmegna P, Garelli F, et al. Artificial Pancreas: Clinical Study in Latin America Without Premeal Insulin Boluses. J Diabetes Sci Technol. 2018; 12(5). doi:10.1177/1932296818786488
25. Steiner S, Hompesch M, Pohl R, et al. A novel insulin formulation with a more rapid onset of action. Diabetologia. 2008; 51(9):1602-1606. doi:10.1007/s00125-008-1095-8
26. Hövelmann U, Heise T, Nosek L, Sassenfeld B, Thomsen K M D, Haahr H. Pharmacokinetic Properties of Fast-Acting Insulin Aspart Administered in Different Subcutaneous Injection Regions. Clin Drug Investig. 2017; 37(5):503-509. doi:10.1007/s40261-017-0499-y
27. Andersen G, Meiffren G, Lamers D, et al. Ultra-rapid BioChaperone Lispro improves postprandial blood glucose excursions vs insulin lispro in a 14-day crossover treatment study in people with type 1 diabetes. Diabetes, Obes Metab. 2018; 20(11):2627-2632. doi:10.1111/dom.13442
28. Senior P, Hramiak I. Fast-acting insulin aspart and the need for new mealtime insulin analogues in adults with type 1 and type 2 diabetes: A Canadian perspective. Can J Diabetes. 2019:1-9.
29. Visentin R, Campos-Náñez E, Schiavon M, et al. The UVA/Padova Type 1 Diabetes Simulator goes from single meal to single day. J Diabetes Sci Technol. 2018; 17(2):237-281.
30. Dalla Man C, Raimondo D M, Rizza R A, Cobelli C. GIM, simulation software of meal glucose-insulin model. J Diabetes Sci Technol. 2007; 1(3):323-330.
31. Schiavon M, Dalla Man C, Cobelli C. Modeling subcutaneous absorption of fast-acting insulin in type 1 diabetes. IEEE Trans Biomed Eng. 2018; 65(9):2079-2086.
32. Andersen G, Meiffren G, Ranson A, Seroussi C, Gaudier M. Ultra-rapid BioChaperone insulin Lispro (BC-LIS): Linear dose-response and faster absorption than insulin Lispro (LIS). Diabetes. 2015; 64(S1):A248.
33. Andersen G, Meiffren G, Lamers D, et al. Ultra-rapid BioChaperone Lispro improves postprandial blood glucose excursions vs insulin lispro in a 14-day crossover treatment study in people with type 1 diabetes. Diabetes, Obes Metab. 2018; 20(11):2627-2632. doi:10.1111/dom.13442
34. Garcia-Tirado J, Colmegna P, Corbett J, Ozaslan B, Breton M D. In Silico Analysis of an Exercise-Safe Artificial Pancreas With Multistage Model Predictive Control and Insulin Safety System. J Diabetes Sci Technol. 2019; 13(6):1054-1064.
35. Clarke W, Kovatchev B. Statistical tools to analyze continuous glucose monitor data. Diabetes Technol Ther. 2009; 11(Suppl 1):S45-S54.
36. Patek S D, Lv D, Ortiz E A, et al. Empirical representation of blood glucose variability in a compartmental model. In: Prediction Methods for Blood Glucose Concentration. Springer; 2016:133-157.
37. Boiroux D, Duun-Henriksen A K, Schmidt S, et al. Overnight glucose control in people with type 1 diabetes. Biomed Signal Process Control. 2018; 39:503-512.
38. Hughes C S, Patek S D, Breton M D, Kovatchev B P. Hypoglycemia prevention via pump attenuation and red-yellow-green “traffic” lights using continuous glucose monitoring and insulin pump data. J Diabetes Sci Technol. 2010; 4(5):1146-1155. doi:10.1177/193229681000400513

What is claimed is:

1. A method of artificial pancreas (AP) control for attaining normoglycemia following an unannounced meal of a subject, comprising:
for a selected insulin, determining at least a corresponding absorption level;
based on a corresponding duration of insulin action (DIA) in dependence on the at least a corresponding absorption level, adjusting the control to change, from a datum, at least a first control parameter penalizing insulin deviation from basal rate and at least a second control parameter representing a difference between two consecutive insulin infusions; and
in response to a detected increase in glucose, infusing the selected insulin in accordance with the adjusted control.

2. The method of claim 1, wherein:
in response to the selected insulin comprising a corresponding absorption level that is higher as against a corresponding absorption level for a non-selected insulin, the at least a first control parameter decreases and the at least a second control parameter increases.

3. The method of claim 2, further comprising:
increasing the infusing based on the at least a first control parameter and the at least a second control parameter.

4. The method of claim 1, wherein:
the infusing the selected insulin is performed to align insulin and meal rates of appearance.

5. A system of artificial pancreas (AP) control for attaining normoglycemia following an unannounced meal of a subject, comprising:
a processor;
a processor-readable memory including processor-executable instructions for:
for a selected insulin, determining at least a corresponding absorption level;
based on a corresponding duration of insulin action (DIA) in dependence on the at least a corresponding absorption level, adjusting the control to change, from a datum, at least a first control parameter penalizing insulin deviation from basal rate and at least a second control parameter representing a difference between two consecutive insulin infusions; and
in response to a detected increase in glucose, infusing the selected insulin in accordance with the adjusted control.

6. The system of claim 5, wherein:
in response to the selected insulin comprising a corresponding absorption level that is higher as against a corresponding absorption level for a non-selected insulin, the at least a first control parameter decreases and the at least a second control parameter increases.

7. The system of claim 6, wherein:
the instructions further comprise increasing the infusing based on the at least a first control parameter and the at least a second control parameter.

8. The system of claim 5, wherein:
the infusing the selected insulin is performed to align insulin and meal rates of appearance.

9. A non-transient computer-readable medium having stored thereon computer-readable instructions for artificial pancreas (AP) control for attaining normoglycemia following an unannounced meal of a subject, said instructions comprising instructions causing a computer to:
for a selected insulin, determining at least a corresponding absorption level;
based on a corresponding duration of insulin action (DIA) in dependence on the at least a corresponding absorption level, adjusting the control to change, from a datum, at least a first control parameter penalizing insulin deviation from basal rate and at least a second control parameter representing a difference between two consecutive insulin infusions; and
in response to a detected increase in glucose, infusing the selected insulin in accordance with the adjusted control.

10. The medium of claim 9, wherein:
in response to the selected insulin comprising a corresponding absorption level that is higher as against a corresponding absorption level for a non-selected insulin, the at least a first control parameter decreases and the at least a second control parameter increases.

11. The medium of claim 10, wherein:
the instructions further cause the computer to perform the infusing based on the at least a first control parameter and the at least a second control parameter.

12. The medium of claim 9, wherein:
the infusing the selected insulin is performed to align insulin and meal rates of appearance.
